(19) United States
(12) Patent Application Publication
(10) Pub. No.: US 2015/0112250 A1
(43) Pub. Date: Apr. 23, 2015

(54) SOLUBLE MICRONEEDLE ARRAYS FOR BUCCAL DELIVERY OF VACCINES

(71) Applicant: TheraJect, Inc., Fremont, CA (US)

(72) Inventor: Sung-Yun Kwon, Fremont, CA (US)

(21) Appl. No.: 14/386,033

(22) PCT Filed: Apr. 3, 2013
(86) PCT No.: PCT/US13/35101

§ 371 Date: Sep. 18, 2014

Related U.S. Application Data
(60) Provisional application No. 61/619,623, filed on Apr. 3, 2012.

(51) Int. Cl. A61M 37/00; A61B 17/20

(52) U.S. Cl. CPC A61M 37/0015; A61B 17/205; A61M 2037/0046; A61B 2017/00004
USPC 604/46

(57) ABSTRACT

A buccal microneedle patch may be provided for vaccination. The buccal microneedle patch may include at least one of microneedles. The at least one microneedles may be configured to contain a predetermined vaccine and to penetrate an outside layer of a buccal mucosa for promptly delivering the predetermined vaccine.

### TECHNICAL FIELD

The present disclosure relates to a dissolving microneedle array and, in particular, to a dissolving microneedle array for oral cavity like buccal vaccination.

### BACKGROUND ART

Vaccination is a cost-effective way to prevent infectious disease. Many new vaccines, including protein, polysaccharides, and deoxyribonucleic acid (DNA), have been developed for better and efficient treatments for vaccination and therapeutic purposes. The most common delivery method for vaccination is a parenteral injection, but the needle injection generally causes needle phobia, unpleasant pain, and local damage to skin and muscle. Additionally, the syringe-needle injection might lead to risk of infection with hepatitis B and C, human immunodeficiency virus (HIV), and other viruses unless needles are disposed of after a single use and unless care is taken to prevent accidental needle sticking of a health care provider. Each year, an estimated 1 billion injections are administered in the United States, and up to 30% of these injections are thought to be unsafe. Other delivery is nasal vaccination. The nasal vaccination, however, is inefficient, low bioavailability and relatively expensive and potentially harmful like sudden facial paralysis (Bell's palsy).

An alternative and safe vaccination target is a mucosal layer. The mucosal layer carries antigen-presenting cells such as Langerhans cells (LCs). In the mucosal layer, LCs makes up 1% of the total cell population, but they have a large spatial extent comprising 25% of the cell surface area. In addition, skin or buccal tissue is relatively easily accessible, has a large surface area (approximately 18,000 cm²), and readily recovers from minor injury. Therefore, skin and buccal has been considered to be an ideal alternative vaccination site.

One of the skin vaccination techniques is a transdermal vaccine delivery or a noninvasive vaccine delivery, such as a gel or patch. The transdermal vaccine delivery or the noninvasive vaccine delivery usually relies on the diffusion of the drug across the skin. Cholera toxin and heat-labile enterotoxin of E. coli penetrate a stratum corneum of mice and humans in sufficient quantities to elicit an immune response. The main drawback of the noninvasive immunization technologies is the low efficiency of antigen uptake due to the stratum corneum's impermeability. Up to date, data on the effectiveness of the noninvasive vaccine delivery are not sufficient in large animal models. This situation is same in buccal vaccination even though the permeability of buccal is much higher than that of skin.

A minimally invasive delivery approach is to use a micro-needle array for piercing the stratum corneum of skin. Hollow micro-needles were proposed to provide a pathway for drug transportation. However, making hollow micro-needles is also a complex and expensive process. A metal micro-needle array was offered to resolve the needle fracture problem. However, the metal micro-needle array can deliver only a limited dose because of the capacity of vaccine coating on the metal needles. Additionally, the fabrication of metal needle arrays and the coating of vaccine onto the needles are complex processes, presumably resulting in the increased cost in manufacturing. Furthermore, the metal needle array brings up a disposal issue. Biodegradable polymer micro-needle arrays were proposed. The potential drawback of biodegradable polymer micro-needle arrays is the longer portal period arising from the slow dissolution of the biodegradable polymer. Microneedle application onto skin requires consistent penetration and independent component for patch adhesion.

### DISCLOSURE

### Technical Problem

Therefore, the present disclosure is to solve the problems of the related art as described above and cope with the requirements.

Accordingly, an object of the present disclosure is to provide an oral cavity, especially buccal microneedle patch including a plurality of solid biodegradable microneedles, which is configured to be inserted into a buccal layer and to be dissolved to supply vaccine in proximity to the dendrite cells.

Another object of the present disclosure is to provide a buccal microneedle patch including a plurality of solid biodegradable microneedles each having a mixture of a dissolving matrix material and a predetermined vaccine.

The foregoing and other objects, features, aspects and advantages of the present disclosure will be understood and become more apparent from the following detailed description of the present disclosure. Also, it can be easily understood that the objects and advantages of the present disclosure can be realized by the units and combinations thereof recited in the claims.

### Technical Solution

In accordance with an embodiment of the present disclosure, a microneedle patch may be provided for vaccination through buccal administration. The microneedle patch may include at least one microneedle. The at least one microneedle may be configured to contain a predetermined vaccine and to penetrate an outside layer of a buccal mucosa for promptly delivering the predetermined vaccine.

The at least one microneedle may be configured to penetrate an epithelium layer of the buccal mucosa and to deliver the predetermined vaccine to a lamina propria.

The at least one microneedle may be made of a material dissoluble, biosoluble, and/or biodegradable and providing bio-adhesion to a surface of the buccal mucosa.

The at least one microneedle may be made of at least one of a cellulose, a dextrin, a dextran, a disaccharide, a chitosan, a chitin, and mixtures thereof. The celluloses may include cellulose, sodium carboxymethyl cellulose, hydroxypropyl cellulose, hydroxyethyl cellulose, hydroxypropyl methylcellulose, amylopectin, gelatin, and pullulan. The dextrins may include maltodextrin, cyclodextrin, amylodextrin, icodextrin, yellow dextrin, and white dextrins. The disaccharides may include sucrose, lactose, maltose, trehalose, turanose, and cellobiose.

The at least one microneedle may be made of at least one of a mixture of sucrose and solidum carboxymethyl cellulose, a mixture of mannose and solidum carboxymethyl cellulose, and a mixture of dextrin and trehalose.

The at least one microneedle may have a tapered shape having a widest diameter smaller than about 900 μm.

The microneedle patch may have a circular shape having a diameter of between about 0.5 cm and about 1.5 cm and includes 5 to 400 microneedles.

The at least one microneedle may be configured with an elongated structure sufficiently long to penetrate the outside layer of the buccal mucosa, to deliver the predetermined vaccine to dendrite cells in a lamina propria, and not to penetrate into or past a submucosa.

A length of the at least one microneedle may be in a range of about 200 to 500 μm.

A length of the at least one microneedle may be in a range of about 20 to 900 μm.

A length of the at least one microneedle may be in a range of about 250 to 750 μm

A length of the at least one microneedle may be less than 1000 μm.

The at least one microneedle may include at least one vaccine of Adenovirus, Anthrax, BCG Live, Diphtheria & Tetanus, Pertussis, Polio, Haemophilus, Hepatitis A, B, Human Papillomavirus, Influenza, Japanese Encephalitis, Meningococcal vaccine, Lyme disease, Rabies, Plague, Pneumococcus, Cholera, Vaccinia, Tuberculosis, Rubella, Measles, Mumps, Rotavirus, Tetanus, Typhoid, Yellow fever, and Zoster.

In accordance with another embodiment of the present disclosure, a microneedle patch may be provided for vaccination through buccal administration. The microneedle patch may include a plurality of sections and each section may include at least one microneedle configured to contain a predetermined vaccine and to penetrate an outside layer of a buccal mucosa for promptly delivering the predetermined vaccine.

Microneedles in one section may contain a vaccine different from a vaccine contained in microneedles in another section.

A shape and a length of microneedles in one section may be different from those of microneedles in another section.

A number of microneedles in one section may be different from a number of microneedles in another section.

### Advantageous Effects

A buccal microneedle patch in accordance with at least one embodiment of the present disclosure may provide effective administration for innate and mucosal and/or humoral vaccination. The advantages of such buccal vaccination with the buccal microneedle patch are more effective mucosal immunization as well as humoral immunization, easy administration: no special training is required for administration, no syringe bio-waste: fully dissolving and edible patch without leaving bio-waste, easy and cheap manufacturing: including conventional manufacturing, inexpensive storage: no need freezer for storage, and simple transportation: inexpensive package and no requirement for cold chain. In addition, the buccal vaccination using the buccal microneedle patch is applicable for cancer vaccine and allergy treatment depending on the immunogen.

### DESCRIPTION OF DRAWINGS

FIG. 1 is a cross-sectional view of a buccal mucosa.

FIG. 2A is a cross-sectional view of a buccal microneedle patch in accordance with at least one embodiment of the present disclosure.

FIG. 2B is a scanning electron microscopic (SEM) image of a buccal microneedle patch in accordance with at least one embodiment of the present disclosure.

FIG. 3 is a perspective view of a buccal microneedle patch in accordance with at least one embodiment of the present disclosure.

FIG. 4A, FIG. 4B, and FIG. 4C shows a buccal microneedle patch applied on oral mucosa 100 in accordance with at least one embodiment of the present disclosure.

FIG. 5 shows a mold used for fabricating microneedles of buccal microneedle patch in accordance with at least one embodiment of the present disclosure.

FIG. 6 shows a fabrication method for a buccal microneedle patch in accordance with at least one embodiment of the present disclosure.

### BEST MODE

Reference will now be made in detail to embodiments of the present disclosure, examples of which are illustrated in the accompanying drawings, wherein like reference numerals refer to like elements throughout. The embodiments are described below, in order to explain the present disclosure by referring to the figures.

The practice of the present disclosure will employ, unless otherwise indicated, conventional methods of engineering, chemistry, biochemistry, pharmacology, and drug delivery, within the skill of the art. Such techniques are explained fully in the literature. All publications, patents and patent applications cited herein, whether supra or infra, are hereby incorporated by reference in their entirety.

A case in which any one part is connected with the other part includes a case in which the parts are directly connected with each other and a case in which the parts are indirectly connected with each other with other elements interposed therebetween. In addition, unless explicitly described otherwise, “comprising” any components will be understood to imply the inclusion of other components but not the exclusion of any other components.

It must be noted that, as used in this specification and the appended claims, the singular forms “a”, “an” and “the” include plural referents unless the content clearly dictates otherwise.

In accordance with at least one embodiment of the present disclosure, a buccal microneedle vaccine patch may be provided for effectively administrating vaccines via a buccal tissue. Such a buccal microneedle patch may include solid biodegradable microneedles. The buccal microneedle patch may be fabricated by mixing vaccine in combination with at least one of solid excipients. The buccal microneedle patch may be configured to allow buccal administration, oral cavity administration, and/or trans-oral administration of predetermined drug. Accordingly, the buccal microneedle patch may effectively inject active ingredients of predetermined vaccines into a immune cell like dendrite cell, macrophage. For example, the buccal microneedle patch may allow injection of vaccines through an oral mucosa by buccal administration and oral cavity administration in accordance with at least one embodiment of the present disclosure. After penetrating the buccal tissue, the buccal microneedle patch including solid biodegradable microneedles dissolves in situ and releases the antigens to the immune system. The vaccine can be any antigen or pathogen which can induce the immune protection or tolerance. The buccal microneedle patch may be referred to as a microneedle, a microneedle array, a microneedle system, a microneedle solid solution microneedle (SSP) patch, and/or a microneedle SSP system.

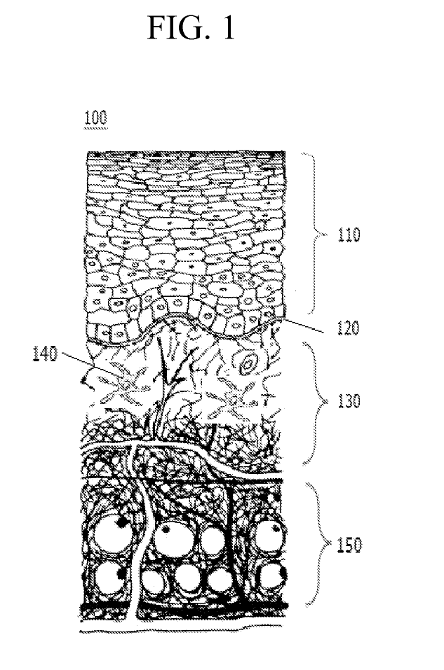

Hereinafter, a buccal microneedle patch and a method for vaccination using the buccal microneedle patch in accordance with at least one embodiment of the present disclosure will be described with reference to FIG. 1 to FIG. 4. As described, the buccal microneedle patch in accordance with at least one embodiment of the present disclosure is configured for a buccal administration and/or an oral cavity administration of a predetermined vaccine. That is, the buccal microneedle patch may be applied on oral mucosa to administer predetermined vaccines. The buccal administration and/or the oral cavity administration of the buccal microneedle patch will be described with reference to FIG. 1.

FIG. 1 is a cross-sectional view of a buccal mucosa.

The buccal mucosa is mucous membranes lining an inside of a mouth. The buccal mucosa may be referred to as an oral mucosa. As shown in FIG. 1, buccal mucosa 100 may include epithelium 110, lamina poria 130, and submucosa 150. Buccal mucosa 100 may further include basement membrane 120 between epithelium 110 and lamina propria 130. The lamina poria 130 may include antigen present cells (APC) 140. For example, such APC 140 may be dendrite cells.

Epithelium 110 may be a stratified squamous layer. Epithelium 110 may include a permeability barrier at the outermost portion thereof. A thickness of such a permeability barrier is about 200 μm. The permeability barrier may be a result of intercellular material derived from the so-called ‘membrane coating granules’ (MCG).

Aside from the permeability barrier such as the MCGs, basement membrane 120 between epithelium 110 and lamina poria 130 may be an additional permeability barrier that acts as resistance to permeation as well. The outer epithelium, however, is still considered to be the rate limiting step to mucosal penetration. The structure of basement membrane 120 is typically not dense enough to exclude even relatively large molecules and is more tolerable to microneedle insertion than skin.

At least one biodegradable microneedle of the buccal microneedle patch in accordance with at least one embodiment of the present disclosure may penetrate the permeability barriers of buccal mucosa 100 and form a channel to deliver a predetermined vaccine contained therein to dendrite cells 140 in lamina propria 130. Accordingly, the buccal microneedle patch is effective method for vaccination. Epithelium 110 generally contains no blood vessels and nerve ending so a patient may not feel pain or bleed, clinically. Once the vaccine dissolves, such epidermis freely exchanges metabolites by diffusion to and from submucosa 150 for further immunization. Submucosa 150 may be located immediately below lamina propria 130. Submucosa 150 may be referred to as a dermis or a dermal layer. The thickness of the dermis is about 1 to 3 mm. Submucosa 150, such as dermis, contains blood vessels, lymphatics, and nerves.

As described, the buccal microneedle patch in accordance with at least one embodiment of the present disclosure is applied on a predetermined area of buccal mucosa for vaccination. That is, the buccal microneedle patch including solid biodegradable microneedles may be provided for administrating vaccines via buccal tissue. The buccal microneedle patch may penetrate epithelium 110 of buccal mucosa 100, form a channel to lamia poria 130, and deliver a predetermined vaccine or drug to immune cell including dendrite cells 140 in lamia poria 130.

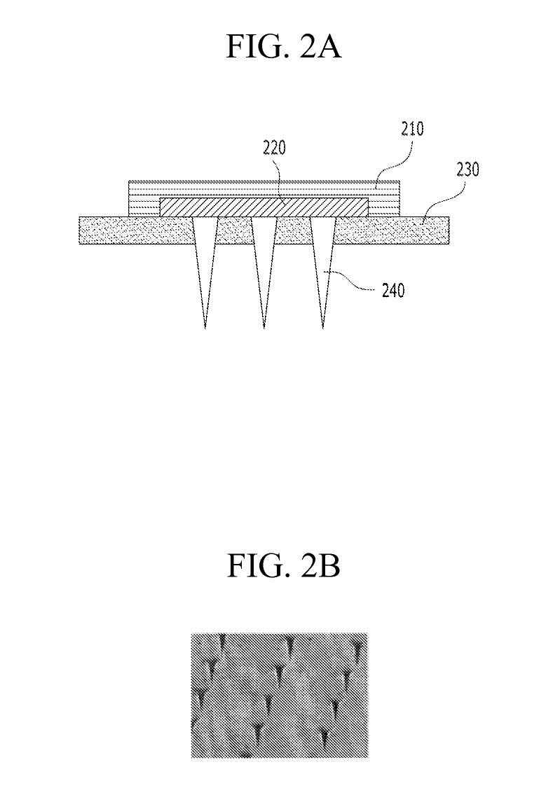

FIG. 2A is a cross-sectional view of a buccal microneedle patch in accordance with at least one embodiment of the present disclosure. FIG. 2B is a scanning electron microscopic (SEM) image of a buccal microneedle patch in accordance with at least one embodiment of the present disclosure.

Referring to FIG. 2A, buccal microneedle patch 200 may include optional backing layer 210, basal layer 220, and a plurality of microneedles 240 in accordance with at least one embodiment of the present disclosure. As shown, buccal microneedle patch 200 may include at least one solid dissolvable or biodegradable microneedle 240, which may be referred to as a perforator. Each one of the solid biodegradable microneedle 240 may include a mixture of a dissolving matrix material and a predetermined vaccine. Such buccal microneedle patch 200 may be used to deliver a antigen or pathogen to subject via their oral cavity like buccal. Accordingly, buccal microneedle patch 200 may improve an immune response.

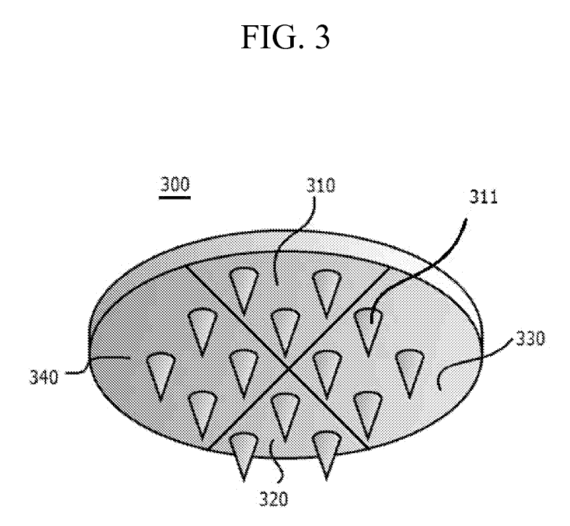

Buccal microneedle patch 200 may have an area of about 2 cm². For example, buccal microneedle patch 200 may be bigger than about 1 cm² and smaller than about 2 cm². Buccal microneedle patch 200 may have a circular shape as shown in FIG. 3. In this case, buccal microneedle patch 200 may have a diameter of between about 0.5 cm and about 1.5 cm. Such circular buccal microneedle patch 200 may have about 5 to 400 solid biodegradable microneedles 240 in an area of about 1 cm². The present disclosure, however, is not limited thereto. Buccal microneedle patch 200 may have various shapes such as a triangle, a rectangle, a polygon, and so forth. Furthermore, the number of microneedles in a unit area may vary according to various factors such as a purpose thereof, a type of a vaccine, the number of vaccines in single buccal microneedle patch 200, and a desired vaccine release rate thereof. For example, about 5 to 500 of microneedles may be included in about 1 cm² of single buccal microneedle patch. More preferably, about 10 to 100 of microneedles may be included. Furthermore, buccal microneedle patch 200 may be a certain size, and composition as described, but the present disclosure is not limited thereto. Such shape, a size, a composition, and an areal density of buccal microneedle patch 200 may affect a vaccine release rate. Accordingly, the shape, the size, the composition, and the areal density of buccal microneedle patch 200 may vary according to a desired vaccine release rate.

Solid biodegradable microneedle 240 may contain predetermined vaccine in accordance with at least one embodiment of the present disclosure. For example, vaccine may be contained in microneedle 240. The primary functions of microneedle 240 may be to pierce the outside of layered epithelium 110, to provide prompt initiation vaccine delivery, and to adhere to the oral cavity tissue until microneedle 240 and/or buccal microneedle patch 200 is completely dissolved and all vaccine in microneedle 240 and/or buccal microneedle patch 200 is delivered. Microneedle 240 may help keep a channel open for subsequent vaccine delivery until a micro channel is closed and a portal channel likely will contract or expand depending on material properties of microneedle 240 after solid biodegradable microneedle 240 and/or buccal microneedle patch 200 dissolves or swells.

Solid biodegradable microneedle 240 may be formed as a solid matrix. Solid biodegradable microneedle 240 may be strong and intact enough to penetrate a subject's buccal tissue, for example, piercing an outside squamous stratified epithelium layer. That is, solid biodegradable microneedle 240 may have sufficient compression strength and keep the sharpness to penetrate human buccal tissue. Such solid biodegradable microneedle 240 may be made of a solid matrix material that is dissoluble, biosoluble, or biodegradable. Furthermore, microneedle 240 may be made of a solid matrix material providing instant bio-adhesion to the oral cavity tissue. Accordingly, microneedle 240 and/or buccal microneedle patch 200 may start to dissolve when microneedle 240 has penetrated into the target tissue, such as the oral cavity tissue. The matrix material of microneedle 240 and/or buccal microneedle patch 200 may be metabolized to give harmless end-products. Microneedle 240 and/or buccal microneedle patch 200 may commence to dissolve immediately after applying buccal microneedle patch 200. For example, within about 10 seconds, microneedle 240 and/or buccal microneedle patch 200 may commence to dissolve after applying buccal microneedle patch 200. Microneedle 240 and/or buccal microneedle patch 200 may be continuously dissolved until microneedle 240 and/or buccal microneedle patch 200 has fully dissolved. For example, microneedle 240 and/or buccal microneedle patch 200 may be dissolved in between a few tens of seconds and several hours, such as up to 1 minutes, 5 minutes, 10 minutes, 20 minutes, 30 minutes, 1 hours, 5 hours, 10 hours or 24 hours.

As long as microneedle 240 and/or buccal microneedle patch 200 dissolves reasonably quickly and is strong enough to pierce the epithelium, fundamentally any biocompatible material may serve as a material for microneedle 240 and/or buccal microneedle patch 200. For example, suitable matrix materials for microneedle 240 and/or buccal microneedle patch 200 may be dissoluble, biosoluble, and biodegradable polymers. Particularly, the suit matrix materials may include a cellulose, a dextrin, a dextran, pectin, a saccharide, a chitosan, a chitin, and the mixtures thereof. Furthermore, generally recognized as safe (GRAS) materials may be also used.

The suitable celluloses may include, but are not limited to, cellulose, sodium carboxymethyl cellulose, hydroxypropyl cellulose, hydroxyethyl cellulose, and hydroxypropyl methylcellulose. The suitable dextrins may include, but are not limited to, maltodextrin, cyclodextrin, amylodextrin, icodextrin, yellow dextrin, and white dextrins. The suitable disaccharides may include, but are not limited to, sucrose, lactose, maltose, trehalose, turanose, and cellobiose. Specially, glucan or derivatives like beta glucan and mannose which is located in bacteria surface membrane may be added as immune stimulant for activating innate immune system. In accordance with at least one embodiment of the present disclosure, suitable mixtures for forming solid biodegradable microneedles 240 and buccal microneedle patch 200 may include a mixture of sucrose and solidum carboxymethyl cellulose, a mixture of mannose and sodium carboxymethyl cellulose, and a mixture of dextrin and trehalose. The sodium carboxy methyle cellulose (Na-CMC) may be preferred to be used as matrix material for forming solid biodegradable microneedles 240 because Na-CMC is inert and provides muco-adhersion properties.

Solid biodegradable microneedle 240 may have a sharpened end for perforating on an oral cavity tissue, for example buccal mucosa 100. Microneedle 240 may have at least one of shapes of a straight shaft, a tapered shaft, a pyramid, a wedge, a needle, a cone, a blade, and so forth. The present disclosure, however, is not limited thereto. In accordance with at least one embodiment of the present disclosure, microneedle 240 may be tapered with a buccal tissue-facing point a shaped as pyramids or cones. Such a tapered microneedle may have a widest diameter smaller than about 900 μm. For example, the number of microneedles included in a single buccal microneedle patch may vary. For example, such a single microneedle patch may include less than 10, 20, 30, 40, 50, 60, 70, 80, 90, 100, 200, 300, 400, 500, 750, 1000 microneedles or more microneedles. Such microneedles may be arranged in a regular repeating pattern or may be arranged irregularly.

Solid biodegradable microneedles 240 penetrate the outmost layer of buccal tissue in accordance with at least one embodiment of the present disclosure. Microneedle 240 may have an elongated structure that is sufficiently long to penetrate through epithelium 110 of the buccal tissue to deliver predetermined vaccine under epithelium 110 and into lamina propria 130. Furthermore, microneedle 240 may have a length not to penetrate into or past submucosa 150. A buccal tissue is not a smooth and rugged surface and has different depths microscopically. In addition, the thickness of epithelium 110 and elasticity of the oral cavity tissue varies. A desirable penetration depth has a range, rather than a single value, for effective vaccine delivery and for painless and bloodless penetration. The penetration depth of microneedle 240 may affect pain as well as delivery efficiency. In accordance with at least one embodiment of the present disclosure, the penetration depth of microneedle 240 may be less than 1000 μm. Accordingly, microneedle 240 may not contact nerves and blood vessels. For example, the length of microneedle 240 may be in a range of about 20 to 900 μm. Particularly, the length of each microneedle 240 may be in a range of about 250 to 750 μm. Preferably, the length of each microneedle 240 may be in a range of about 200-500 μm.

As shown in FIG. 2A, buccal microneedle patch 200 may further include basal layer 220 and backing layer 210 in accordance with at least one embodiment of the present disclosure.

Basal layer 220 may provide instant mucosal adhesion to the oral cavity tissue. Furthermore, basal layer 220 may provide a reservoir of vaccines or drugs in accordance with at least one embodiment of the present disclosure. The function of basal layer 220 is adhesion to the oral cavity tissue and provide extra drug for sustained delivery. A thickness of basal layer 220 may vary. Where additional and sustained vaccine release is required, basal layer 220 may be constructed to contain more of vaccine or drug. Such a contained vaccine or drug may be different from vaccine contained in microneedle 240, but the present disclosure is not limited thereto. In some embodiments, basal layer 220 may contain the same vaccine as that contained in microneedle 240.

Backing layer 210 may be formed on basal layer 220 in accordance with at least one embodiment of the present disclosure. For example, backing layer 210 may cover at least one outer side of basal layer 220. Backing layer 210 may provide protection of microneedle 240 from local saliva and tongue movement. Backing layer 210 may contain flavor and color components to mask medicine taste. Backing layer 210 may be made of material that is a dissolvable and edible matrix, but the present disclosure is not limited thereto. In some embodiment, backing layer 210 may be formed of un-dissolvable matrix depending on application.

Backing layer 210 may be formed of material different from that of microneedle 240 and/or basal layer 220 in order to help microneedle 240 and/or basal layer 220 to fully dissolve. For example, backing layer 210 may be formed of material dissolving slowly than microneedle 240 and basal layer 220. Backing layer 210 may be prepared by at least one of direct compression, dry granulation, and wet granulation. After forming backing layer 210, backing layer 210 may be combined with basal layer 220 and microneedle 240. For example, backing layer 210 may be bonded on a base layer of microneedle 240. In addition, vaccine may be loaded on backing layer 210 to simulate gamma and delta T cell receptors on tongue.

As described, buccal microneedle patch 200 in accordance with at least one embodiment of the present disclosure is very effective for vaccination. For example, other intradermal needle formats have been found to be incompatible with the high level of residual detergent that can be present in surface antigen influenza vaccines. Unlike the other intradermal needle formats, solid biodegradable microneedles 240 of buccal microneedle patch 200 are even effective in these circumstances.

Particularly, buccal microneedle patch 200 in accordance with at least one embodiment of the present disclosure is applicable for virus particles (i.e., virion) consisting of genetic materials such as deoxyribonucleic acid (DNA) or ribonucleic acid (RNA), a protein coat for protecting the genes, and an envelope of lipids surrounding the protein coat when they are outside a cell. Accordingly, such virus particles may be prepared through a splitting process for clarification of virion-containing material in order to remove non-virion material and an adsorption method for concentration of the harvested virions. As the adsorption method, CaHPO₄ adsorption may be used.

Furthermore, buccal microneedle patch 200 may be applicable for virosomes. The virosomes are nucleic acid free viral-like liposomal particles. The virosomes can be prepared through solubilization of virus with a detergent after removal of the nucleocapsid and reconstitution of the membrane containing the viral glycoproteins. An alternative method for preparing virosomes may involve adding viral membrane glycoproteins to excess amounts of phospholipids, to give liposomes with viral proteins in their membrane. Live attenuated viruses are obtained from viruses (grown in eggs or in cell culture), but the viruses are not inactivated.

In accordance with at least one embodiment of the present disclosure, buccal microneedle patch 200 may be applicable for following vaccines: Adenovirus, Anthrax, BCG Live, Diphtheria & Tetanus, Pertussis, Polio, Haemophilus, Hepatitis A, B, Human Papillomavirus, Influenza, Japanese Encephalitis, Meningococcal vaccine, Lyme disease, Rabies, Plague, Pneumococcus, Cholera, Vaccinia, Tuberculosis, Rubella, Measles, Mumps, Rotavirus, Tetanus, Typhoid, Yellow fever, Zoster, Other DNA vaccine, and other vaccine. For example, microneedles 240 of buccal microneedle patch 200 may include at least one of following vaccines: Adenovirus, Anthrax, BCG Live, Diphtheria & Tetanus, Pertussis, Polio, Haemophilus, Hepatitis A, B, Human Papillomavirus, Influenza, Japanese Encephalitis, Meningococcal vaccine, Lyme disease, Rabies, Plague, Pneumococcus, Cholera, Vaccinia, Tuberculosis, Rubella, Measles, Mumps, Rotavirus, Tetanus, Typhoid, Yellow fever, Zoster, Other DNA vaccine, and other vaccine.

In accordance with at least one embodiment of the present disclosure, buccal microneedle patch 200 may be formed of a sugar based solid form. Accordingly, buccal microneedle patch 200 may have a design for developing multivalent vaccines easily and be more thermostable. Furthermore, vaccination through buccal microneedle patch 200 may need less dose of a predetermined vaccine compared to typical syringe dose because an oral immune system is a part of extensive mucosa-associated lymphoid tissue (MALT). In addition, cytokines, chemokines, or adjuvant may be added in vaccine formulation.

Buccal microneedle patch 200 was described as one vaccine. The present disclosure, however, is not limited thereto. In some embodiments, single buccal microneedle patch 200 may include multiple vaccines. Such example of buccal microneedle patch 200 will be described with reference to FIG. 3.

FIG. 3 is a perspective view of a buccal microneedle patch in accordance with at least one embodiment of the present disclosure.

As shown in FIG. 3, buccal microneedle patch 300 may have a circular shape, but the present disclosure is not limited thereto. Furthermore, buccal microneedle patch 300 may have a plurality of sections 310 to 340, each having at least one microneedle 311 having a vaccine different from that included in microneedles of the other section. For example, a vaccine included in microneedles in first section 310 is different from that included in microneedles in second section 320.

Accordingly, single buccal microneedle patch 300 may have multiple vaccines without mixing different vaccines together in single microneedle in accordance with at least one embodiment of the present disclosure. That is, single buccal microneedle patch 300 may be used for multiple different vaccinations.

Each section of buccal microneedle patch 300 may have a different number of microneedles. Furthermore, microneedles in each section of buccal microneedle patch 200 may have a shape, a size, and a length different from those included in each other section of buccal microneedle patch 300. Such the number, the shape, the size and the length may be determined according to various factors including a type of vaccines, the number of vaccines, a required vaccine release rate, and so forth.

In addition, buccal microneedle patch 300 is illustrated as having four sections 310 to 340 in FIG. 3. The present disclosure, however, is not limited thereto. The number of sections in single buccal microneedle patch may vary according to various factors.

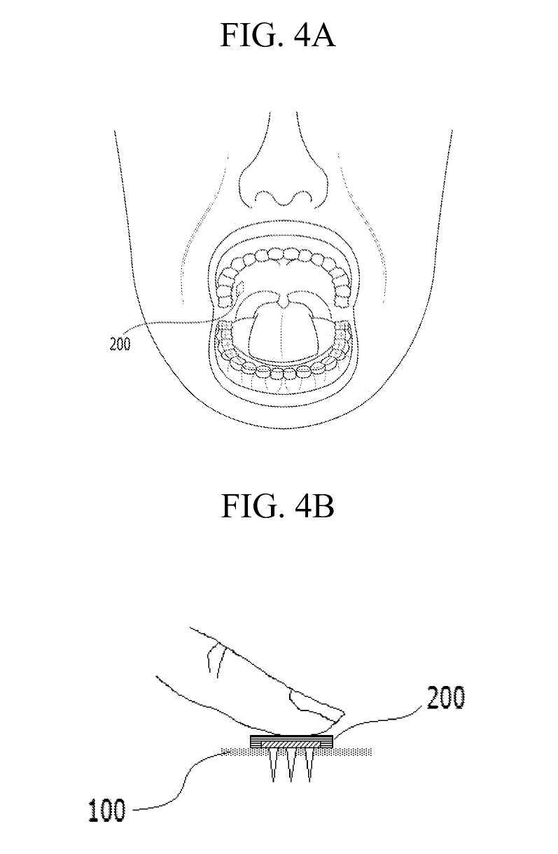

FIG. 4A, FIG. 4B, and FIG. 4C shows a buccal microneedle patch applied on oral mucosa 100 in accordance with at least one embodiment of the present disclosure.

As shown in FIG. 4A to FIG. 4C, buccal microneedle patch 200 in accordance with at least one embodiment of the present disclosure may be placed on a patient's mouth and holding it in the mouth, either adjacent a cheek and/or between the upper lip and gum. That is, buccal microneedle patch 200 may deliver a predetermined vaccine contained in at least one of microneedles 240 through buccal administration. Buccal microneedle patch 200 begins to dissolve or disintegrate due to the moisture in the mouth and saliva. Such a delivery of vaccine via oral cavity such as buccal and sublingual is a very effective for achieving systemic or vaccination effects.

Buccal microneedle patch 200 may be used to deliver a vaccine to a subject via buccal 100. For example, buccal microneedle patch 200 may be suitable for administering vaccines to human subjects. Buccal microneedle patch 200 may raise an immune response in a subject. Such immune response may include an antibody response, preferably a protective antibody response.

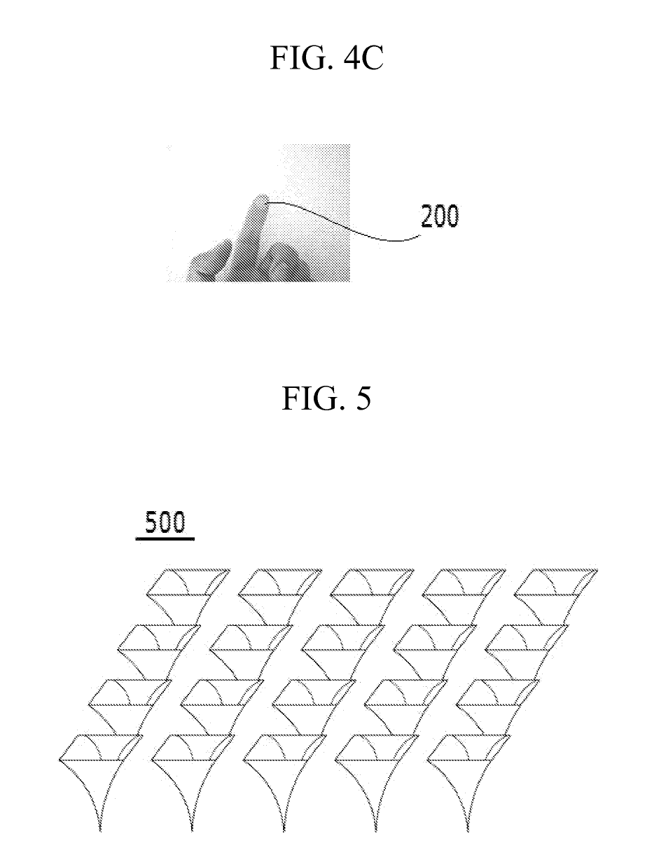

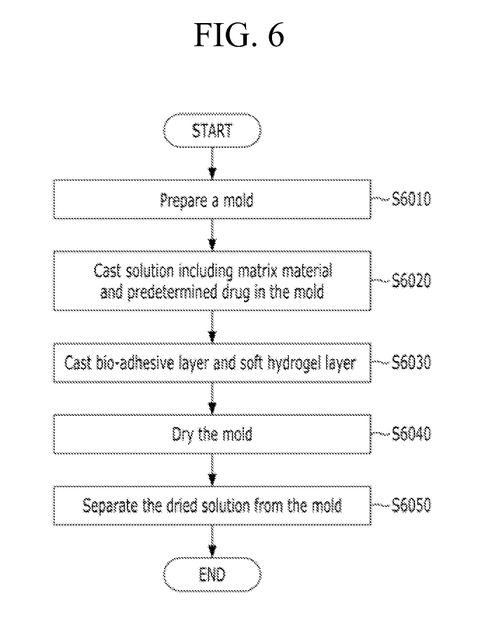

FIG. 5 shows a mold used for fabricating microneedles of buccal microneedle patch in accordance with at least one embodiment of the present disclosure and FIG. 6 shows a fabrication method for a buccal microneedle patch in accordance with at least one embodiment of the present disclosure.

Referring to FIG. 5 and FIG. 6, a mold or a micro mold for buccal microneedle patch may be prepared at step S6010. For example, mold 500 of FIG. 5 may be prepared through precision machining such as milling, micro-machining (such as MEMS), laser-based machining, and electro-discharge machining. Particularly, such mold 500 may include about microneedle cavities each having a specific length such as about 500 μm. For a description of a representative mold, see e.g., U.S. patent application Ser. No. 13/364,438, filed Feb. 2, 2012, incorporated herein by reference in its entirety.

At step S6020, solution including a matrix material and a predetermined drug may be cast in the mold and dried. The solution may be at least one of liquid, gel solution, and melted sugar. The predetermined drug may be at least one of vaccines including Adenovirus, Anthrax, BCG Live, Diphtheria & Tetanus, Pertussis, Polio, Haemophilus, Hepatitis A, B, Human Papillomavirus, Influenza, Japanese Encephalitis, Meningococcal vaccine, Lyme disease, Rabies, Plague, Pneumococcus, Cholera, Vaccinia, Tuberculosis, Rubella, Measles, Mumps, Rotavirus, Tetanus, Typhoid, Yellow fever, Zoster, Other DNA vaccine, and other vaccine. The present disclosure, however, is not limited thereto.

Depending on the viscosity and other physical and chemical properties of the solution, additional force such as centrifuge force or compression force may be applied to fill the mold.

For example, a matrix material of dextrin and trehalose may be combined with a predetermined vaccine/adjuvant. Such aqueous material may be centrifugally cast in the mold to form solid biodegradable microneedles 240.

At step S6030, a bio-adhesive layer and a soft hydrogel layer may be sequentially cast after the filling. For example, the bio-adhesive layer may be basal layer 220 and the soft hydrogel may be backing layer 210. Depending on the viscosity and other physical and chemical properties of the liquid solution, additional force such as centrifuge force or compression force may be applied. Particularly, a cellulose gel may be cast over the matrix/vaccine film to form backing layer 210 thereon.

At step S6040, the mold may be dried to form a solid solution. For example, in order to dry, at least one of air dry, vacuum dry, and freeze dry may be applied.

At step S6050, once the solution is completely dried, the dried solution may be separated from the mold and cut to an appropriate shape and size for oral cavity administration. The shape and size may vary according to a desired drug release rate. In accordance with at least one embodiment of the present disclosure, a size of the buccal microneedle patch may be about 1 to 2 cm².

Individual or multiple buccal microneedle patches may be packaged into individual or group pouches, respectively. For example, individual or group pouches may be sealed under nitrogen with heat.

A powder form may be used for the material for buccal microneedle patch 200. In this case, a mixed powder may be spread over the mold. For example, the mixed power may include a predetermined drug particle. Depending upon chemical and physical properties of the mixed powder, a direct compression process, a wet granulation process, and a heating process may be applied to melt the mixed power and to insert viscous material into the mold. Alternatively, the mixed powder may be inserted into the mold by pressure and/or application of heating with use of binding agents.

Solid biodegradable microneedles 240 of buccal microneedle patch 200 may be prepared using one of direct compression, dry granulation, and wet granulation. The direct compression, the dry granulation, and the wet granulation may be utilized for preparation of the mix prior to a compression stage. The direct compression may be used for fabricating a microneedle with a powder form of ingredients which can be mixed well and do not require further granulation steps prior to introduction to the microneedle negative mold for pressing. The dry granulation may be used for the blending of the ingredients followed by compaction and size reduction of the mix in order to produce a granular, free flowing blend of uniform size the compacting process. In addition, it is important to evenly distribute a vaccine through microneedles. If this cannot be done simply through adequate blending, the ingredients can go through an additional granulation step prior to the compression step of the press in order to ensure an even distribution of the API in the final tablet. The wet granulation involves the production of a granule by the addition of liquid binders to the powder mixture. Both continuous direct compression (CDC) and continuous mixing for the dry granulation processes involve the individual loading and accurate feeding of the API and a variety of excipients to a continuous blender. In addition, lubricants (e.g. magnesium stearate) are added to the mix to improve powder flow so that the die of the tablet press fills accurately.

As described above, although the present disclosure is described by the limited embodiments and drawings, the present disclosure is not limited to the above-described. Various forms of substitutions, modifications and alterations may be made by those skilled in the art from the above description without departing from the spirit of the prevent invention.

Accordingly, the scope of the present disclosure is not construed as being limited to the described embodiments but is defined by the appended claims as well as equivalents thereto.

### INDUSTRIAL APPLICABILITY

A buccal microneedle patch in accordance with at least one embodiment of the present disclosure may be used for effectively mucosal and/or humoral vaccination.

1. A microneedle patch for vaccination through buccal administration, the microneedle patch comprising:
at least one microneedle configured to contain a predetermined vaccine and to penetrate an outside layer of a buccal mucosa for promptly delivering the predetermined vaccine.

2. The microneedle patch of claim 1, wherein the at least one microneedle is configured to penetrate an epithelium layer of the buccal mucosa and to deliver the predetermined vaccine to a lamina propria.

3. The microneedle patch of claim 1, wherein the at least one microneedle is made of a material dissoluble, biosoluble, and/or biodegradable and providing bio-adhesion to a surface of the buccal mucosa.

4. The microneedle patch of claim 1, wherein:
the at least one microneedle is made of at least one of a cellulose, a dextrin, a dextran, a disaccharide, a chitosan, a chitin, gelatin, amylopectin, pullulan and mixtures thereof;
the celluloses include cellulose, sodium carboxymethyl cellulose, hydroxypropyl cellulose, hydroxyethyl cellulose, and hydroxypropyl methylcellulose;
the dextrins include maltodextrin, cyclodextrin, amylodextrin, icodextrin, yellow dextrin, and white dextrins; and
the disaccharides include sucrose, lactose, maltose, trehalose, turanose, and cellobiose.

5. The microneedle patch of claim 1, wherein the at least one microneedle is made of at least one of a mixture of sucrose and solidum carboxymethyl cellulose, a mixture of mannose and solidum carboxymethyl cellulose, and a mixture of dextrin and trehalose.

6. The microneedle patch of claim 1, wherein the at least one microneedle has a tapered shape having a widest diameter smaller than about 900 μm.

7. The microneedle patch of claim 1, wherein the microneedle patch has a circular shape having a diameter of between about 0.5 cm and about 1.5 cm and includes 5 to 400 microneedles.

8. The microneedle patch of claim 1, wherein the at least one microneedle is configured with an elongated structure sufficiently long to penetrate the outside layer of the buccal mucosa, to deliver the predetermined vaccine to dendrite cells in a lamina propria, and not to penetrate into or past a submucosa.

9. The microneedle patch of claim 1, wherein a length of the at least one microneedle is in a range of about 200 to 500 μm.

10. The microneedle patch of claim 1, wherein a length of the at least one microneedle is in a range of about 20 to 900 μm.

11. The microneedle patch of claim 1, wherein a length of the at least one microneedle is in a range of about 250 to 750 μm.

12. The microneedle patch of claim 1, wherein a length of the at least one microneedle is less than 1000 μm.

13. The microneedle patch of claim 1, wherein the at least one microneedle includes at least one vaccine of Adenovirus, Anthrax, BCG Live, Diphtheria & Tetanus, Pertussis, Polio, Haemophilus, Hepatitis A, B, Human Papillomavirus, Influenza, Japanese Encephalitis, Meningococcal vaccine, Lyme disease, Rabies, Plague, Pneumococcus, Cholera, Vaccinia, Tuberculosis, Rubella, Measles, Mumps, Rotavirus, Tetanus, Typhoid, Yellow fever, and Zoster.

14. A microneedle patch for vaccination through buccal administration, the microneedle patch comprising:
a plurality of sections, each section including at least one microneedle configured to contain a predetermined vaccine and to penetrate an outside layer of a buccal mucosa for promptly delivering the predetermined vaccine.

15. The microneedle patch of claim 14, wherein microneedles in one section contain a vaccine different from a vaccine contained in microneedles in another section.

16. The microneedle patch of claim 14, wherein a shape and a length of microneedles in one section are different from those of microneedles in another section.

17. The microneedle patch of claim 14, wherein a number of microneedles in one section is different from a number of microneedles in another section.

18. The microneedle patch of claim 14, wherein the at least one microneedle is configured with an elongated structure sufficiently long to penetrate the outside layer of the buccal mucosa, to deliver the predetermined vaccine to dendrite cells in a lamina propria, and not to penetrate into or past a submucosa.

19. The microneedle patch of claim 14, wherein a length of the at least one microneedle is in a range of about 200 to 500 μm.

20. The microneedle patch of claim 14, wherein the at least one microneedle includes at least one vaccine of Adenovirus, Anthrax, BCG Live, Diphtheria & Tetanus, Pertussis, Polio, Haemophilus, Hepatitis A, B, Human Papillomavirus, Influenza, Japanese Encephalitis, Meningococcal vaccine, Lyme disease, Rabies, Plague, Pneumococcus, Cholera, Vaccinia, Tuberculosis, Rubella, Measles, Mumps, Rotavirus, Tetanus, Typhoid, Yellow fever, and Zoster.
